(19) United States
(12) Patent Application Publication
(10) Pub. No.: US 2025/0304761 A1
(43) Pub. Date: Oct. 2, 2025

(54) PROCESS AND ACTIVE CATALYST FOR THE GLYKOLYSIS OF POLYETHYLENE TEREPHTHALATE (PET)

(71) Applicant: Rewin Textiles AB, Malmö (SE)

(72) Inventors: Henrik WENE, Malmö (SE); Niklas JAKOBSSON, Kågeröd (SE)

(73) Assignee: Rewin Textiles AB, Malmö (SE)

(21) Appl. No.: 18/864,986

(22) PCT Filed: May 10, 2023
(86) PCT No.: PCT/EP2023/062488

§ 371 Date: Nov. 12, 2024

(30) Foreign Application Priority Data
May 12, 2022 (SE) 2250571-3

(51) Int. Cl. C08J 11/24; B01J 35/40; B01J 35/58; B01J 35/61; B01J 35/63

(52) U.S. Cl. CPC C08J 11/24; B01J 35/40; B01J 35/58; B01J 35/613; B01J 35/615; B01J 35/633; B01J 35/635; C08J 2367/02

(57) ABSTRACT

The invention pertains to a method for catalytic glycolysis of polyethylene terephthalate (PET), comprising the steps of a) mixing PET with ethylene glycol (EG) in a reactor with a catalytic filter, catalysing depolymerisation of PET, and heating the mixture such that the PET is depolymerised, whereby forming a reaction mixture comprising bis(2-Hydroxyethyl) terephthalate (BHET) and PET oligomers, b) cooling the resulting reaction mixture from step a), whereby precipitating BHET and oligomers, and c) separating precipitated BHET and oligomers, at least partly, from unreacted EG, wherein the catalytic filter comprises a transesterification catalyst catalysing depolymerisation of PET, the catalyst being immobilized on a fiber material. Also is provided a catalytically active filter for catalytic depolymerisation of PET, a method of producing a catalytically active filter and a reactor system for catalytic glycolysis of polyethylene terephthalate (PET).

### FIELD OF THE INVENTION

This invention pertains in general to the field of catalytic de-polymerization of used polymers into their respective monomers for further processing and utilization. More specifically, the invention pertains to an active catalyst product configuration, a reactor design and a process layout and a method to produce monomeric polymer building blocks through catalytic glycolysis reactions. As such, the invention pertains to a process to prepare very high purity Bis(2-Hydroxyethyl) terephthalate (BHET) using polyethylene terephthalate (PET) as the starting product, which may be recovered from waste, using non-toxic products, such as ethylene glycol and water.

### BACKGROUND OF THE INVENTION

It is known that plastic waste is one of the big problems that will have to be faced during the coming decades. Nearly 300 million tonnes of plastic wastes are produced every year. The problem is that 75% of all plastic produced has become waste, much will be released into nature, and that it takes around 500-1,000 years for plastics to decompose. The plastic waste problem is further complicated by processes in nature forming so called micro-plastics, plastic particles so small that they are taken up by the biosphere, where they are feared to cause unknown toxic effects.

Therefore, it is important to recover plastic before it is released into nature. One way to recover plastic is by burning it, recovering the plastic as heat, and releasing most of the remaining material as gaseous waste products (i.e. CO₂). However, it is even better if the plastic may be recycled as new materials, preferably many times, before it is finally destroyed (i.e. heat recycled).

Polyethylene Terephthalate (PET) is the most common thermoplastic polymer resin of the polyester family and is used in fibers for clothing, containers for liquids and foods, and thermoforming for manufacturing, and in combination with glass fiber for engineering resins. Further foamed PET is used as a lightweight construction material. PET is well known, for instance through use as food containers, such as so-called PET bottles.

While PET collected or separated into fractions with very high purity may be directly re-used using mechanical recycling, the degree of polymerization and purity will inherently be lowered in recycled PET affecting its properties. Such sorting and collecting also results in that a large fraction of total used PET is not recovered but rather found in the reject flow of mixed plastics. Eventually, further mechanical re-cycling is not possible and alternative re-cycling of at least the monomers would be desirable. Preferably, PET wastes are recycled, such as it can be reused again, for instance through chemical recycling. Chemical recycling methods of PET include chemical processes such as acidic or basic hydrolysis, methanolysis, or glycolysis to provide for recycling of the monomers in PET.

In EP0723951A1, is shown such a process to prepare Bis(2-Hydroxyethyl) terephthalate (BHET), here through glycolysis, where waste PET reacts with excess ethylene glycol in the presence of a transesterification catalyzer and the BHET is recovered through crystallization from an aqueous solution. This method is mild and uses using non-toxic products, such as ethylene glycol and water.

However, although several different chemical recycling plants have been started, it has been hard to get a cost-effective chemical recycling process. One problem that is faced is impurities in the recycling stream, contaminants and degradation products generated during processing, which both causes problems for the chemical recycling process, and may result in lower quality recycled materials.

As such, there is a need for efficient methodologies and strategies for chemical recycling methods for polymers, such as recycled PET, resulting in high purity recycled materials without producing effluents that are environmentally harmful and/or difficult to treat and allowing for maximum recycling and re-use of hydrocarbon materials.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a method for catalytic glycolysis of polyethylene terephthalate (PET), comprising the steps of: a) mixing PET with ethylene glycol (EG) in a reactor with a catalytic filter, catalysing depolymerisation of PET, and heating the mixture such that the PET is depolymerised, whereby forming a reaction mixture comprising bis(2-Hydroxyethyl) terephthalate (BHET) and PET oligomers, b) cooling the resulting reaction mixture from step a), whereby precipitating BHET and oligomers, and c) separating precipitated BHET and oligomers, at least partly, from unreacted EG, wherein the catalytic filter comprises a transesterification catalyst catalysing depolymerisation of PET, the catalyst being immobilized on a fiber material.

Also is provided a catalytically active filter for catalytic depolymerisation of PET, comprising a catalyst fused to a fiber material in the form of a filter, such that the catalyst is immobilized, wherein the fiber material is selected from the group consisting of a metal fiber, sintered metal fiber, carbon fiber, ceramic fiber, a aluminia silicate based fiber, an alumina fiber, a glass fiber a PTFE fiber, a P84 fiber, and the catalyst comprises a carrier with an high internal surface area such as Alumina, Titania, Ceria, Zirconia or mixtures thereof, and a catalytically active metal such as Cu, Mn, Fe, Zn, Mg, Na, K, oxides of Cu, Mn, Fe, Zn, mg, Na, K, acetates such as K(OAc)₂, Zn(OAc)₂, Na₂CO₃ or mixtures thereof, or the fiber material is a porous ceramic fiber or an alumina silicate fiber with high internal surface area, and the catalyst is a catalytically active metal selected from Cu, Mn, Fe, Zn, Mg, Na, K, oxides of Cu, Mn, Fe, Zn, mg, Na, K, acetates such as K(OAc)₂, Zn(OAc)₂ and Na₂CO₃ or mixtures thereof.

Further is provided a method of producing a catalytically active filter for catalytic depolymerisation of PET, comprising the steps of: a) making a fiber material catalytic by adhering a catalyst onto a fiber surface of the fiber material, and b) fusing the catalyst onto the fiber surface to create a catalyst that is immobilized, wherein the fiber material is a metal fiber, sintered metal fiber, carbon fiber, ceramic fiber, a aluminia silicate based fiber, an alumina fiber, a glass fiber a PTFE fiber, or a P84 fiber; and/or the catalyst comprises a carrier with an high internal surface area such as Alumina, Titania, Ceria, Zirconia or mixtures thereof and a catalytically active metal such as Cu, Mn, Fe, Zn, Mg, Na, K, oxides of Cu, Mn, Fe, Zn, Mg, Na, K, acetates such as K(OAc)₂, Zn(OAc)₂, Na₂CO₃ or mixtures thereof, and the catalyst is fused onto the fiber surface by heat treatment, or the fiber material is a porous ceramic fiber or an alumina silicate fiber, and the catalyst is a metal catalyst or metal catalyst precursor and is impregnated directly onto the porous ceramic fiber.

Also, is provided a reactor system for catalytic glycolysis of polyethylene terephthalate (PET), the reactor system comprising at least one depolymerization vessel, wherein the depolymerization vessel comprises at least one feed inlet for feeding PET and EG to the vessel, at least one outlet for withdrawing BHET and oligomers from the vessel, and at least one catalytic filter being arranged downstream of the inlet and upstream of the outlet, wherein the catalytic filter comprises a bound transesterification catalyst for catalysing depolymerisation of PET, the catalyst being immobilized on a fiber material.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic representation of the method of the invention, where (1) PET and EG is placed in the first reactor comprising the active catalytic filter system where the glycolysis of PET takes place in parallel with the removal of insoluble materials, BHET is purified in subsequent steps of (2) removing residual insoluble materials and oligomers (3) final BHET purification through re-crystallizations and (4) polymerization of PET and recovery of EG;

FIG. 2 provides a schematic view of the PET glycolysis reaction;

FIG. 3 provides a schematic view of a PET glycolysis reactor of the invention with a stationary fiber section;

FIG. 4 provides a schematic view of a reactor system of the invention with catalytic filter sections and ports for particulates removal as well as for addition of additives in the chambers in-between the catalytic filter chambers. The chambers in-between the filter sections are designed with sufficient space to allow addition of additives and removal of particulates as well as providing a mixing zone to equalize the flow maldistribution that may arise at the outlet of one filter section before the flow of liquid flow continues into the following filter section. Additives can act to remove colorants, improve particulate removal, improve dissolving rates, oxidize impurities and remove colorants. Such additives are known in the field and may include flocculation additives, additives to increase the dissolving rate, oxidation agents such as ozone, hydrogen peroxide and absorption materials such as activated carbon to remove colorants or combinations thereof. Furthermore, one or several of the filter sections may have different mesh size in each filter section providing removal of smaller particles of insoluble materials and additional residence time for larger particles containing PET; and

FIG. 5 provides a schematic view of a PET glycolysis reactor of the invention where the fiber based catalyst is disparaged in the liquid phase and thus constituting a slurry bed reactor with a mechanical filter to separate fiber from liquid at the reactor outlet.

### DESCRIPTION OF EMBODIMENTS

The following description focuses on an embodiment of the present invention applicable to an active catalyst product configuration, a reactor design and a process layout that together provides a method to produce monomeric polymer building blocks through catalytic glycolysis reactions.

In the invention, it was realized that many of the problems faced during chemical recycling processes are linked to two main challenges. The first challenge is that the process requires a catalyst that is sufficiently active, overcomes the mass transfer limitations inherent to a liquid reaction system and does not contaminate the product or the effluent streams. A homogeneous catalyst can provide sufficient catalytic activity and overcome mass transfer limitations and be separated from the product through crystallization. However, a homogeneous catalyst will inevitably end up contaminating the water or EG effluent streams or both. Using a traditional heterogenous catalyst will require that the size of the catalyst is in the micro or nano meter range which makes it difficult to recycle such catalyst when used in glycolysis of waste plastic feedstock where the catalyst material will mix with non-PET polymer and non-polymer residues. Furthermore, impure starting material and mechanically different size of the starting material leading to different temperatures and uneven reaction conditions, all of this resulting in less efficient chemical recycling and less purity of the recycled final products.

It was hypothesized that several of these problems could be solved if one could use a fiber supported catalyst that is a fixed structure in the glycolysis reactor providing a large exposed catalyst surface to overcome external mass transfer resistance, a fiber diameter in the micrometer range to reduce mass transfer resistance within the material and since the catalyst is immobilized in the reactor it will not contaminate product or effluent streams. Furthermore, constituting the fiber material as a filter bed and with a flow of reactants and product through the filter in the reaction chamber, a residence time distribution is accomplished for non-dissolved fragments leading to a longer residence time i.e. reaction time for larger fragments compared to smaller fragments and dissolved PET molecules.

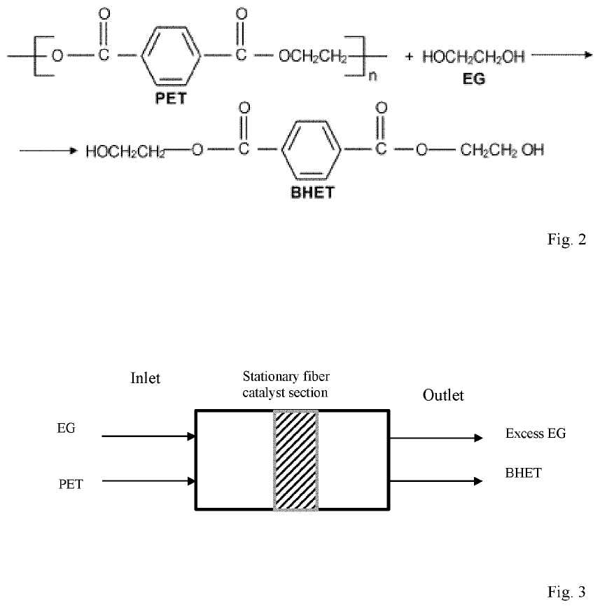

To prove the concept, a glycolysis process to process PET to BHET, such as described in EP0723951A1 and shown in FIG. 2, was used, wherein waste PET reacts with excess EG in the presence of a transesterification catalyst. This method is mild and uses using non-toxic products, such as EG and water.

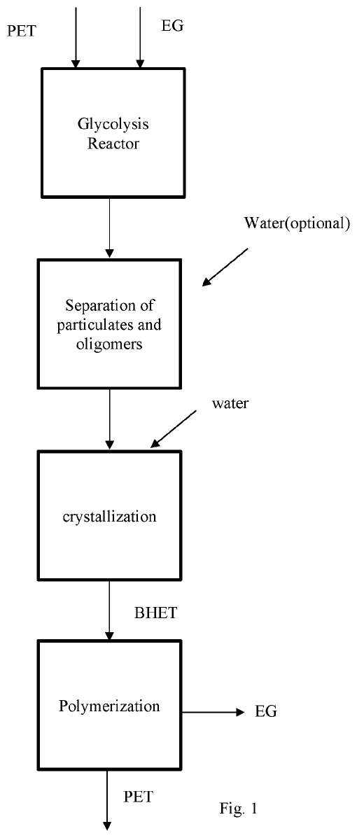

During trials, an active catalytic filter material was developed that ensured selection of suitable PET fragments for active catalysis, here for glycolysis of PET to BHET. A full reaction overview from recycled PET to re-polymerized PET is shown in FIG. 1.

The filter provides a 3D mesh network where the active catalyst is bound. The filter thus both sorts the reaction start materials (such as recycled PET particles) by size, as well as ensuring that the correct materials may react selectively, i.e. inside the active filter material. Once reaction has been performed, small end-products (i.e. the resulting monomeric polymer building blocks, e.g., BHET) may exit the filter, and as such, not stay in the active catalytic environment longer than necessary. This helps prevent unfavorable side reactions of the reactants and products.

The following filter characteristics was found to be most suitable for such a reaction:

The diameter of the fiber material is a trade-off between maximizing outer surface, minimizing internal diffusion length and mechanical strength. The active catalytic filter should preferably constitute a filter bed or multitude of filter beds where the catalysed fiber material has a diameter of 5-200 micrometer diameter, preferably 5-50 micrometer and most preferably 5-10 micrometer diameter.

The fiber material can be a metal fiber, sintered metal fiber, carbon fiber, ceramic fiber, an aluminia silicate based fiber, an alumina fiber, a glass fiber a PTFE fiber, a P84 fiber or similar. The fiber is catalyzed by adhering a catalyst material to the fiber and then binding or fusing the catalyst onto the filter surface at elevated temperature. The catalytic material comprises a carrier with an high internal surface area such an alumina, Titania, Ceria, Zirconia or mixtures thereof and a catalytically active metal such as Cu, Mn, Fe, Zn, mg, Na, K, oxides of Cu, Mn, Fe, Zn, mg, Na, Na, K, acetates such as K(OAc)₂, Zn(OAc)₂, Na₂CO₃ or mixtures thereof.

The catalysed fiber can also be a porous ceramic fiber or an alumina silicate fiber, preferably with high internal surface area, supporting and immobilizing a catalytically active component or mix of components. The catalysed fiber is directly impregnated with a catalytically active metal such as Cu, Mn, Fe, Zn, mg, Na, K, oxides of Cu, Mn, Fe, Zn, mg, Na, Na, K, acetates such as K(OAc)₂, Zn(OAc)₂ and Na₂CO₃ or mixtures thereof and subsequently dried and treated at elevated temperature (i.e. heat treated) to adhere or fuse the catalyst to the fiber material.

The porous ceramic fiber or an alumina silicate fiber with high internal surface area may have a surface area of 20 cm²/g or higher, such as 20 to 800 cm²/g.

Such porous ceramic fiber material may have a surface area of between 20 to 280 m²/g and a pore volume of 0.05 to 0.8 cm³/g.

The most preferred catalyst materials comprise a carrier of primarily alumina and an active metal oxide of ZnO or Fe₂O₃, or mixtures thereof.

The catalyst may be fused to the fiber material.

The treatment at elevated temperature (i.e. heat treatment to bind or fuse the catalyst onto the fiber surface) preferably takes place at a temperature of between 200 and 600 degrees Celsius.

Some catalysts, such as oxides of Cu, Mn, and Fe may require an additional treatment at elevated temperature in a calcination and decomposition step to create the active catalyst component. The required temperature for such calcination and decomposition will depend on the specific catalyst species but a temperature between 20° and 600° C. will be required.

The catalytic filter fiber material in the form of a woven fibers, felted fibers, fibers shaped using a webbing process or fibers put together using vacuum forming together with a binder.

In the invention is shown a method of producing a catalytically active filter for catalytic depolymerisation of a PET polymer, comprising the steps of a) making a fiber material catalytic by adhering a catalyst onto a fiber surface of the fiber material, and b) fusing the catalyst onto the fiber surface to create a catalyst that is immobilized.

The method to produce the catalytically active filter may further comprise a step c) of forming the fiber material to the form of a filter. Additionally, the fiber material may be arranged to constitute a catalytic filter reactor.

The fiber based catalyst configuration creates a catalyst product that is immobilized and thus not contaminating the product or the waste water and glycol streams in the process. This can in principle also be accomplished by using pellet or fragment based heterogenous catalyst. However due to the significant mass transfer resistance for such catalyst structures, studies show that micrometer and nanometer size particulates are necessary (see Journal of Cleaner Production, 225 (2019) 1052-1064. Section 2.5.4 and table 6; Yonghwan Kim et. al., Polymers 2022, 14, 656. https://doi.org/10.3390/polym14040656) and thus separating such catalyst particles from the liquid implies significant technical challenges as well as detrimental cost implications for the process. The fiber based catalyst product configuration creates a catalyst that is immobilized and thus does not need to be separated from the liquid, has a large outer surface to overcome external mass transfer limitations between fiber and the bulk liquid phase, a mass transfer diffusion length of a few micrometre or less and does not contaminate water or EG effluents from the process.

During trials with the active filter catalyst, it was found that the filter allowed for new reactor designs as well as modified PET glycolysis processes, which were co-developed in the invention.

In the invention, a process where BHET was produced through Catalytic glycolysis of PET using the active fiber based filter catalyst. The catalytic filter comprises a transesterification catalyst catalysing depolymerisation of PET, the catalyst being immobilized on a fiber material. The process consists of a number of steps:
a) PET is mixed with Ethylene glycol (EG) and heated to the appropriate temperature. The mixture is mixed in or fed into a reactor and contacted with the fiber-based catalyst. The mixture is heated such that the PET is depolymerised, whereby forming a reaction mixture comprising bis(2-Hydroxyethyl) terephthalate (BHET) and PET oligomers.

The EG is preferably in a stoichiometric excess.

A preferred ratio between PET and EG is 1:3 to 1:9, a more preferred ratio is 1:3.7-1:6, and a most preferred ratio is between 1:4-1:5.

The preferred operation temperature is between 150-300° C., more preferred temperature 180 to 280° C., most preferred between 190-260° C.

The resulting reaction mixture from step a) may be filtered to remove oligomers and non-PET material. The filtering might be performed by the catalytic filter and/or by an additional filter material. As such, the catalytic filter may prevent insoluble particles, such as dirt and non-polyester components, from entering the downstream process (i.e. step b).
b) The liquid phase is cooled, preferably to an ambient temperature, whereby BHET and oligomers are precipitated.
c) precipitated BHET and oligomers are separated, at least partly, from unreacted EG, for instance by filtering.

The BHET and PET oligomers of step c) may be separated by any suitable filtering method, such as filtering or centrifuging.

The separated solid from step c) may be solubilized in water, followed by precipitation, re-crystallization and filtering at specific temperatures, to obtain BHET with high purity.

In step b), the reaction mix may be cooled by the addition of water to a temperature of between 6° and 90° C., preferably between 65 and 75° C. The amount of water added, which may affect the ease with which the subsequent stages of the process occur and the overall cost of the BHET, may vary with the reaction mix in a ratio of 1:0.1 to 1:10 (mass/mass) and preferably between 1:0.5 to 1:2. The temperature control during this phase is important, since the BHET must solubilize in the aqueous solution, while the oligomers (which otherwise co-crystallise with the BHET, preventing the formation of sufficiently large BHET crystals and contaminating it), must remain in suspension.

During separation of the oligomers in step c), the aqueous solution is cooled slowly to precipitate out the BHET the water solubility of which varies with the temperature. To obtain a high yield of BHET, the final temperature must reach between −10 to +30° C., and preferably between 5 to 15° C.

The BHET crystals are separated from the aqueous solution, which contains most of the excess EG, through filtering or centrifuging. The solid recovered is dissolved again in hot water until reaching a temperature of 70-100° C. and then cooled to a temperature of between 0 to 30° C., preferably between 5 to 15° C., to obtain high-purity BHET crystals that are easy to filter.

In this second crystallisation phase, the ratio of the amount of water to BHET ranges between 1:4 and 1:10, and preferably between 1:6 and 1:8. BHET in the form of crystals is recovered by filtering and further polymerized to form PET and EG. The produced EG can be sold or recycled to the glycolysis process.

During development, it was found that not only did the method work, but trial experiments benchmarked against current state-of-the-art methods showed a better BHET yield when using the method of the invention, as is shown in Example 1.

In the glycolysis process to process PET to BHET, the PET material is a solid that is solvated by the EG and thus after solvatisation, PET, EG reactants and the BHET product constitutes a homogeneous liquid. This liquid can thus be pumped through a reactor loaded with the fiber-based catalyst.

The method could be operated without flow, such as batch-wise in a closed reactor where the product and reactants come into contact with the fiber based catalyst through natural or forced convection within the reactor. Such forced convection could be accomplished through agitation or by moving the fiber based catalyst through the liquid phase comprising reactants and product, or the product and reactants may flow through a reactor with the fiber-based catalyst inside the reactor.

The reactor can be in the form of a tubular reactor where the fiber-based catalyst is stationary inside the reactor and the product and reactants flow through the reactor i.e. a tubular reactor configuration.

The reactor may comprise at least two catalytic filters, wherein a downstream filter(s) have a different permeability and density and/or a different catalyst formulation than a first, upstream filter.

Similarly, the reactor comprises at least two catalytic filters, wherein subsequent filter(s) downstream of a first filter have the same catalyst formulation as the first filter, but sequentially finer mesh sizes, thereby providing further removal of smaller particles and insoluble materials and additional residence time for larger PET particles and PET-oligomers.

The fiber material can furthermore be in the form of a filter i.e., forming a catalytically active filter.

By utilizing one or several catalytic filters of different permeability and density and potentially with different catalyst formulations, separation of contaminants can be achieved simultaneously with catalytic conversion of PET.

The filter will also provide a longer residence time for larger PET fragments or material where the PET is mixed or fused together with impurities such as in multilayer films and fabrics, which are known to have a lower effective rate of reaction.

In Yonghwan Kim et. al. (Polymers 2022, 14, 656. https://doi.org/10.3390/polym14040656) it is clearly shown that a longer residence time for solvatisation does not negatively impact the yield of BHET. Retaining PET containing materials for a longer time allowing less accessible PET to be dissolved into the reaction liquid will therefore not provide a significant negative impact of the overall yield. This will on the contrary improve the overall yield since more of the PET in the feed material will be subject to glycolysis and conversion to BHET. This enables a glycolysis process that is significantly more efficient than current techniques for processing of waste PET.

In the invention. a reactor system was developed, comprising at least one depolymerization vessel, wherein the depolymerization vessel comprises at least one feed inlet for feeding PET and EG to the vessel, at least one outlet for withdrawing BHET and oligomers (and excess EG) from the vessel, and at least one catalytic filter being arranged downstream of the inlet and upstream of the outlet.

The depolymerization vessel may have two feed inlets for feeding PET and EG to the vessel. Such a system is illustrated in FIG. 3.

The depolymerization vessel may be in a form of a reactor where the catalytic filter is stationary inside the reactor and the product and reactants flow through the reactor.

The catalytic filter prevents insoluble materials (e.g. dirt and non-polyester components) from entering the downstream process steps and provides residence time distribution providing larger PET particles and larger particles containing PET but where the other polymers are not dissolved and thus remains in solid phase to be retained while smaller particles flow through the filter more rapidly. This provides a mechanism where the feed material with lower reactivity will receive a longer residence time and feed material with higher reactivity (fragment with high content of PET and/or smaller size) has a shorter residence time i.e. essentially providing a more uniform product distribution, higher yield and less formation of undesired biproducts.

The reactor system may comprise at least two catalytic filters sections with different mesh size in each filter. The catalytic filter(s) may have sequentially finer mesh sizes, thereby providing further removal of smaller particles of insoluble materials and additional residence time for larger particles containing PET.

A reactor system may also comprise at least one catalytic filter section and at least one non-catalytic filter section.

This enables a (less preferred) system design, wherein the catalytic filter section may be made up of a slurry of fibre based catalyst suspended in the reactor, which are subsequently retained in the reactor by filtration of the non-catalytic filter section. During such trials the slurry of fiber based catalyst may form a filter cake the non-catalytic filter section (being downstream of the filter slurry), thus forming two filter sections. An example of such a system can be seen in FIG. 5.

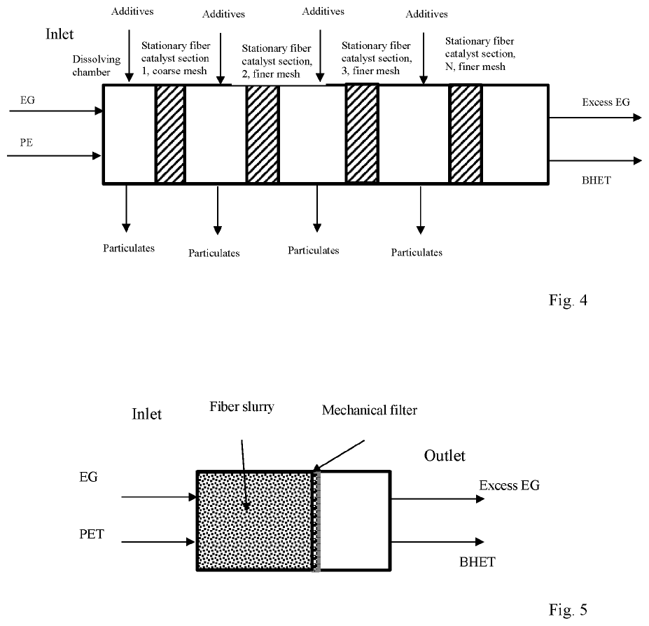

A catalytic filter based reactor may constitute multiple filters sections with different mesh size in each filter. An example of such a setup can be seen in FIG. 4. For example, the coarsest filter mesh may allow particles of a size of up to 2 mm to pass the filter whereas the finest mesh size may only allow particulates of up to 5 micrometres to penetrate the filter. A multitude of filters sections may be installed between the coarsest and finest filter sections and these sections can be designed with mesh sizes allowing particulate material between 2 mm and 5 micrometres to pass through such filter sections.

The fiber material of the catalytic filter section in the reactor can be supported between metal (or other material that can withstand the reaction temperature and chemical composition of the liquid in the reactor) mesh structures to create reactor internals where the fiber material is compressed to reach the desired material bulk density, permeability and residence time.

In one example of the invention, fiber material of the catalytic filter may be dispersed at the feed inlet and collected on a filter cake together with unconverted material. This can also be configured as a multitude of reactors and/or combined with catalytic filters to optimize the BHET yield and removal of impurities.

The desired material bulk density, permeability and residence time can also be achieved by vacuum forming to form rigid structures from the fibrous materials and binding agents which is well known from production of high temperature thermal insulation components for furnaces and other process equipment in for example the glass manufacturing and steel industry.

Heating and/or cooling may be carried out between fiber based catalyst sections to provide optimal conditions for removal of contaminants and/or increased BHET yield.

In the reactor system comprises at least two of catalytic filter sections, preferably additives may be injected in-between the sections and preferably solid material can be removed before or in-between the sections.

Injection of additional EG streams between the fiber based catalyst sections may be carried out to provide optimal conditions for removal of contaminants and/or BHET yield.

Injection of solid adsorbents and/or agglomeration agents and/or precipitation agents between the fiber based catalyst sections may be carried out to remove contaminants before subsequent reaction steps.

Injection of additional catalytic material between the fiber based catalyst sections may be carried out to provide optimal conditions for removal of contaminants and/or BHET yield. Catalytically active materials are Cu, Mn, Fe, Zn, mg, Na, K, oxides of Cu, Mn, Fe, Zn, mg, Na, Na, K, acetates such as K(OAc)₂, Zn(OAc)₂, Na₂CO₃ or mixtures thereof, and other homogenous catalysts known in the field.

It is well known that activated carbon can be used to remove colorants and other contaminants and that various oxidation agents such as H₂O₂, O₃ etc. can be used to decompose contaminants and colorants [WO2021124149A1] and thus these measures can be taken between the sections of fiber based catalyst product or as a post treatment of the liquid.

A reactor design according to the invention may be a tubular reactor with a fixed catalytic internal material which provides a significant improvement in reaction conditions compared to batch reactors, semi batch reactors or continuous tank reactors since optimum conditions for PET/EG ratio as well as optimum catalyst space velocity can be upheld throughout the reactor.

A further advantage compared to utilizing homogeneous catalyst technology is that for a homogeneous catalyst, the reaction continues during the cooling phase between the reactor and the first crystallization step. In this invention the reaction stops when the liquid stream leaves the reactor since no catalyst is present outside the rector

Studies have shown (Yonghwan Kim et. Al. Polymers 2022, 14, 656. https://doi.org/10.3390/polym14040656) that BHET yield has a direct link to the amount of catalyst used i.e. there is an optimum catalyst load and too much catalyst leads to promotion of competing reactions that does not form BHET.

### Example 1—Fiber Based Catalyst vs. State of the Art Catalyst

Experiments were carried out in 150 ml reactors (2 pcs) with 10 g PET (New raw material from Invista “RT20”) and 50 ml ethylene glycol (technical quality 98%). The amount of catalyst in the experiment with the fiber based catalyst product was 20 wt % based on the amount of PET loaded into the reactor. For more efficient mixing, 3 inert ceramic beads (3-4 mm in diameter) were added per reactor.

The reactors were mounted in a rotating holder (30 rpm) in an oven heated to a temperature of 230° C. The time for heating reactors is assumed to be about 10 minutes. Reaction time was set to 60 min.

Separation of monomer was according to standard procedure with filtrations, crystallization and recrystallization.

Measurement of yield (weight BHET) and weights of material collected on filter paper was performed.

Glycolysis was performed with the catalytic filter product and a state of the art catalyst but otherwise the same conditions. The resulting yield can be seen in table 1. As can be seen, the method using the catalytic filter not only worked, but showed a better BHET yield than current state-of-the-art methods.

TABLE 1

| | Reference state of the | | Fiber based | |
|---|---|---|---|---|
| | art catalyst | | catalyst product | |
| | A | B | A | B |
| PET (g) | 10.1 | 10.2 | 10.0 | 10.0 |
| Total loaded solid material (g) | 0.05 | 0.05 | 2.0 | 2.0 |
| Effective cat component (g) | 0.05 | 0.05 | 0.02 | 0.02 |
| EG (ml) | 50 | 50 | 50 | 50 |
| Solid residue (g) | 1.0 | 0.80 | 2.04 | 2.9 |
| BHET yield (g) | 10.1 | 9.4 | 10.1 | 10.2 |
| BHET yield (mole) | 85.2 | 79.9 | 86.2 | 86.6 |

### Example 2—the Impact of the Catalyst Product Configuration and the Reactor Design

The impact of the catalyst product configuration and the reactor design is summarized in table 2 below.

TABLE 2

| Main materials balance | Annual amount | | |
|---|---|---|---|
| PET | 20,000 | ton | Consumed |
| Water | 200,000 | ton | Consumed |
| Ethylene Glycol | 80,000 | ton | Recycled |
| BHET | 20,300 | on | Intermediate product |
| | | | Produced |
| PET | 18,000 | ton | |

For a 20,000 PET recycling plant utilizing this invention will thus enable that the 200,000-ton water stream that is not contaminated by any homogeneous catalyst and that the 80,000-ton Ethylene Glycol is not contaminated with any homogeneous catalyst.

The present invention thus enables a lower cost of purification for the significant effluent streams of water and Ethylene Glycol.

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A method for catalytic glycolysis of polyethylene terephthalate (PET), comprising the steps of:
a) mixing PET with ethylene glycol (EG) in a reactor with a catalytic filter, catalysing depolymerisation of PET, and heating the mixture such that the PET is depolymerised, whereby forming a reaction mixture comprising bis(2-Hydroxyethyl) terephthalate (BHET) and PET oligomers,
b) cooling the resulting reaction mixture from step a), whereby precipitating BHET and oligomers, and
c) separating precipitated BHET and oligomers, at least partly, from unreacted EG,
wherein
the catalytic filter comprises a transesterification catalyst catalysing depolymerisation of PET, the catalyst being immobilized on a fibre material.

2. The method according to claim 1, where the catalyst is fused to the fiber material.

3. The method according to claim 1, wherein the catalytic filter comprises fiber material in the form of a woven fibers, felted fibers, fibers shaped using a webbing process or fibers put together using vacuum forming together with a binder.

4. The method according to claim 1, wherein the fiber material is a porous ceramic fiber or an alumina silicate fiber with high internal surface area supporting and immobilizing a catalytically active component or mix of components.

5. The method according to claim 1, wherein the fiber material is arranged to constitute a catalytic filter reactor.

6. The method according to claim 1, wherein the preferred ratio (wt./wt.) between PET and EG in step a) is between 1:3 to 1:9, preferably between 1:3.7 to 1:6, more preferably between 1:4 to 1:5.

7. The method according to claim 1, wherein the heating of step a) is to a temperature of between 15° and 300° C., preferably between 18° and 280° C., more preferably between 19° and 260° C.

8. The method according to claim 1, wherein in step b), the reaction mixture is cooled by the addition of water to a temperature of between 6° and 90° C., preferably between 65 and 75° C., and the water is added at a mass to mass ratio (reaction mixture:water) of between 1:0.1 and 1:10, more preferably between 1:0.5 and 1:2.

9. The method according to claim 1, wherein the temperature in step c) is −10 to +30° C., preferably 5 to 15° C.

10. The method according to claim 1, wherein the BHET and PET oligomers of step c) are separated by filtering or centrifuging.

11. The method according to claim 1 wherein the separated BHET and PET oligomers from step c) is solubilized in water, followed by precipitation, re-crystallization and filtering or centrifuging, to obtain BHET with high purity.

12. The method according to claim 11, wherein the temperature is between 7° and 100° C. during the solubilisation, and between 0 and 30° C., preferably between 5 and 15° C., during the precipitation and crystallization to obtain high-purity BHET crystals.

13. The method according to claim 11, wherein the ratio (wt./wt.) of water to BHET ranges between 1:4 to 1:10, preferably between 1:6 to 1:8 during the precipitation and crystallization.

14. The method according to claim 1, wherein the catalytic filter prevents insoluble particles, such as dirt and non-polyester components, from entering the downstream process step b).

15. The method according to claim 1, wherein the reactor comprises at least two catalytic filters, wherein a downstream filter(s) have a different permeability and density and/or a different catalyst formulation than a first, upstream filter.

16. The method according to claim 1, wherein the reactor comprises at least two catalytic filters, wherein subsequent filter(s) downstream of a first filter have the same catalyst formulation as the first filter, but sequentially finer mesh sizes, thereby providing further removal of smaller particles and insoluble materials and additional residence time for larger PET particles and PET-oligomers.

17. A catalytically active filter for catalytic depolymerisation of PET, comprising a catalyst fused to a fiber material in the form of a filter, such that the catalyst is immobilized, wherein
the fiber material is selected from the group consisting of a metal fiber, sintered metal fiber, carbon fiber, ceramic fiber, a aluminia silicate based fiber, an alumina fiber, a glass fiber, a PTFE fiber, a P84 fiber, and the catalyst comprises a carrier with an high internal surface area such as Alumina, Titania, Ceria, Zirconia or mixtures thereof, and a catalytically active metal such as Cu, Mn, Fe, Zn, mg, Na, K, oxides of Cu, Mn, Fe, Zn, Mg, Na, K, acetates such as K(OAc)₂, Zn(OAc)₂, Na₂CO₃ or mixtures thereof,
or
the fiber material is a porous ceramic fiber or an alumina silicate fiber with high internal surface area, and the catalyst is a catalytically active metal selected from Cu, Mn, Fe, Zn, Mg, Na, K, oxides of Cu, Mn, Fe, Zn, Mg, Na, K, acetates such as K(OAc)₂, Zn(OAc)₂ and Na₂CO₃ or mixtures thereof.

18. The catalytically active filter according to claim 17, wherein the fiber material has a fiber diameter of 5 to 200 micrometer, preferably 5 to 50 micrometer and most preferably 5 to 10 micrometer.

19. The catalytically active filter according to claim 17, wherein the porous ceramic fiber material has a surface area of between 20 to 280 m²/g and a pore volume of 0.05 to 0.8 cm³/g.

20-24. (canceled)

25. A reactor system for catalytic glycolysis of polyethylene terephthalate (PET), the reactor system comprising at least one depolymerization vessel, wherein the depolymerization vessel comprises
at least one feed inlet for feeding PET and EG to the vessel,
at least one outlet for withdrawing BHET and oligomers from the vessel, and
at least one catalytic filter being arranged downstream of the inlet and upstream of the outlet,
wherein the catalytic filter comprises a bound transesterification catalyst for catalysing depolymerisation of PET, the catalyst being immobilized on a fiber material.

26. The reactor system according to claim 25, wherein the depolymerization vessel comprises two feed inlets for feeding PET and EG to the vessel.

27. The reactor system according to claim 25, wherein the depolymerization vessel is in the form of a reactor where the catalytic filter is stationary inside the reactor and the product and reactants flow through the reactor.

28. The reactor system according to claim 25 wherein the reactor system comprises at least two of catalytic filter sections where additives can be injected in-between the sections and solid material can be removed before or in-between the sections.

29. The reactor system according to claim 25 where the reactor system constitutes at least two catalytic filters sections with different mesh size in each filter.

30. The reactor system according to claim 25 where the reactor system constitutes at least one catalytic filter section and at least one non-catalytic filter section.

31. The reactor system according to claim 25, wherein the fiber based catalyst constitutes a slurry of suspended fibers that are subsequently retained in the reactor by filtration.
